(19) United States
(12) Patent Application Publication
(10) Pub. No.: US 2011/0237948 A1
(43) Pub. Date: Sep. 29, 2011

(54) ULTRASOUND PROBE FOR USE WITH DEVICE PERFORMING NON-CONTACT RESPIRATORY MONITORING

(75) Inventor: Stephen B. CORN, Sharon, MA (US)

(73) Assignee: ENGINEERED VIGILANCE, LLC, Sharon, MA (US)

(21) Appl. No.: 13/079,509

(22) Filed: Apr. 4, 2011

Related U.S. Application Data
(63) Continuation-in-part of application No. 12/363,467, filed on Jan. 30, 2009.
(60) Provisional application No. 61/320,717, filed on Apr. 3, 2010.

(51) Int. Cl. A61B 8/13

(52) U.S. Cl. 600/443

(57) ABSTRACT

An ultrasound probe designed for use with a monitoring system performing non-contact respiratory and cardiac monitoring is described. The ultrasound probe may be used with smart phones and tablet computing devices or standalone monitoring devices to capture respiratory and cardiac function in a monitored subject. The probe may come equipped with a hinged stand and utilize a number of different interface connections to communicate with the monitoring device.

### RELATED APPLICATION

The present application is related to and claims the benefit of U.S. Provisional Patent Application No. 61/320,717, filed Apr. 3, 2010, entitled “Ultrasound Probe for Use With Device Performing Non-Contact Respiratory Monitoring.” The present application is also a continuation-in-part of U.S. application Ser. No. 12/363,467, filed Jan. 30, 2009, and entitled “System and Method Providing Biofeedback for Anxiety and Stress Reduction.” The contents of both applications are incorporated herein by reference in their entirety.

### BACKGROUND

Recently, non-contact monitoring systems using radiated energy to identify cardiac and respiratory waveforms in patients have been developed. See, for example, pending U.S. application Ser. No. 11/308,675, entitled “Method for Using a Non-Invasive Cardiac and Respiratory Monitoring System.” The monitoring system illuminates a subject in radiated energy and then detects the reflected radiated energy caused by respiratory and/or cardiac functions. The detected reflections are used to plot a two-dimensional waveform. The waveforms represent the rise and fall of a detected signal (the reflected energy) over time and are indicative of the small movements of the patient's chest and abdomen that are associated with cardiac and respiratory function. Different monitoring systems may use different portions of the electro-magnetic spectrum such as radio frequencies or laser or ultrasonic energy to capture breathing and cardiac waveforms for analysis. The waveforms may be used to diagnose a number of different types of respiratory and cardiac disorders and to provide biofeedback useful in the treatment of respiratory and cardiac conditions and treatment of stress reduction and anxiety. See, for example, pending U.S. application Ser. No. 12/363,467, entitled “System and Method Providing Biofeedback for Anxiety and Stress Reduction.”

### BRIEF SUMMARY

Embodiments of the present invention provide an ultrasound probe designed for use with a monitoring system performing non-contact respiratory and cardiac monitoring. For example, the ultrasound probe may be used with smart phones and tablet computing devices or standalone monitoring devices to capture respiratory and cardiac function in a monitored subject. The probe may come equipped with a stand that may be hinged and utilize a number of different interface connections to communicate with the monitoring device.

In one embodiment, an ultrasound probe apparatus for use with a portable monitoring system performs non-contact monitoring of respiratory function. The apparatus includes transmission means for transmitting ultrasonic waves in a range above 20 kHz towards a monitored subject. The apparatus also includes receiving means for receiving reflected waves bouncing off of the monitored subject and an interface for transmitting data regarding the transmitted and received ultrasonic waves to a monitoring system executing on a portable device. The portable device is equipped with a processor. The monitoring system includes a waveform detection module for generating a waveform from the data. The portable device also includes, or is in communication with, an analysis module for programmatically analyzing the generated waveform to identify a respiratory function of the subject.

In another embodiment, a portable monitoring system performing non-contact monitoring of respiratory function includes an ultrasound probe transmitting ultrasonic waves in a range above 20 kHz towards a monitored subject. The probe also receives reflected waves bouncing off of the monitored subject. The system also includes an interface for transmitting data regarding the transmitted and received ultrasonic waves to a portable device. The portable device is equipped with a processor and includes a waveform detection module that receives data about the transmitted and reflected waves from the probe and generates a waveform from the data. The portable device includes, or is in communication with, an analysis module for programmatically analyzing the generated waveform to identify a respiratory function of the subject.

In one embodiment, a method for monitoring respiratory function using an ultrasound probe transmits with an ultrasound probe ultrasonic waves in a range above 20 kHz towards a monitored subject and receives with the ultrasound probe reflected waves bouncing off of the monitored subject. The method also transmits from the probe to a portable device the data regarding the transmitted and received ultrasonic waves. The data is transmitted via a probe interface. The method further generates programmatically on the portable device a waveform from the data and analyzes programmatically the generated waveform to identify a respiratory function of the subject.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1 depicts an exemplary environment suitable for practicing embodiments of the present invention;

FIG. 1A depicts an exemplary interface board suitable for use with some embodiments of the present invention;

FIG. 2 depicts an exemplary integrated biofeedback apparatus equipped with an ultrasound probe;

FIG. 3 depicts an exemplary sequence of steps performed by an embodiment of the present invention to gather data with an ultrasound probe;

FIG. 4A depicts an exemplary ultrasound probe;

FIG. 4B depicts an exemplary ultrasound probe with a hinged stand connected to a smart phone; and

FIG. 4C depicts an exemplary ultrasound probe built into a stand for a tablet computing device.

### DETAILED DESCRIPTION

Embodiments of the present invention provide an ultrasound probe designed for use with a non-contact monitoring system that monitors and analyzes physiologic functions of a monitored subject. The non-contact monitoring system is capable of providing biofeedback in real-time to the monitored subject. Non-contact measurement of breathing parameters (e.g.: rate, rhythm, amplitude, pauses, inspiratory to expiratory ratio, breathing frequency variability), and/or cardiac parameters (e.g.: rate, rhythm, amplitude, heart rate variability) and/or body movements are acquired through the use of the ultrasound probe. The captured information is transmitted to the monitoring device for analysis.

The ultrasound probe captures breathing rate, rhythm, amplitude, breathing rate variability and I:E Ratio (Inspiratory to Expiratory Ratio). The probe is able to acquire data from a clothed subject and may include a swivel head for directing and focusing the ultrasound beam. The probe also may come equipped with a stand (described further below) that may be hinged and that is capable of holding an attached smart phone or tablet computing device in position.

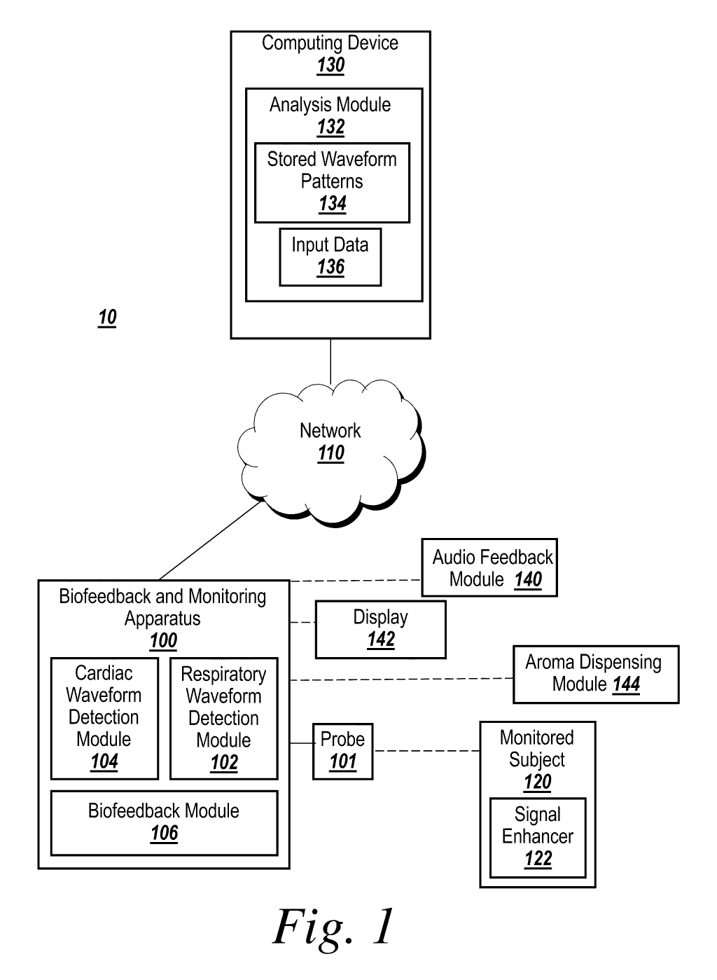

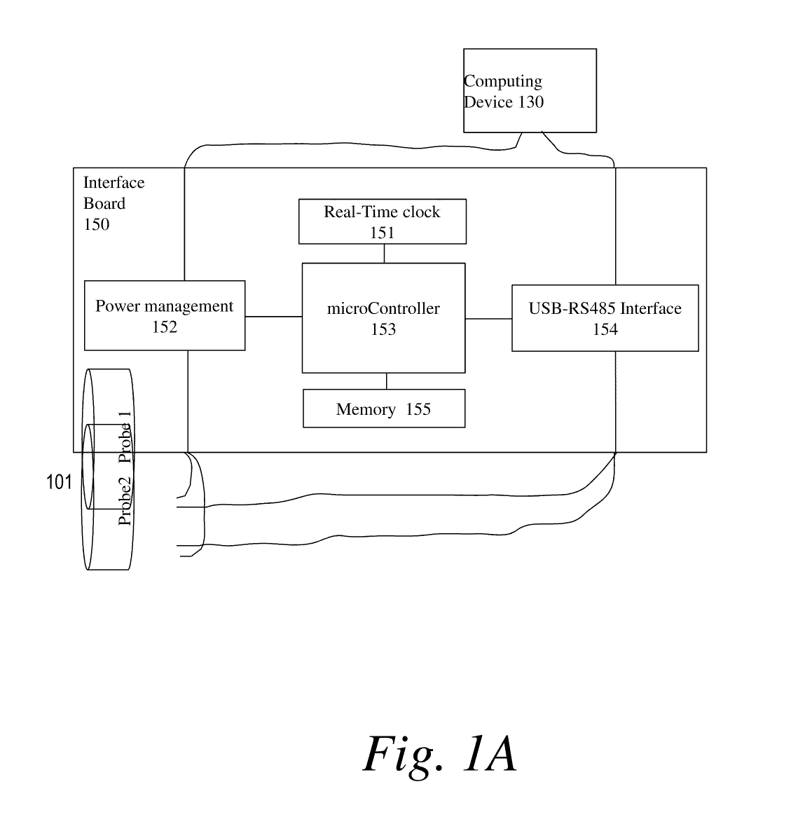

FIG. 1 depicts an exemplary environment suitable for practicing embodiments of the present invention. Biofeedback and monitoring system 10 may include biofeedback and monitoring apparatus 100 that is used to monitor physiological factors for a monitored subject 120. Biofeedback and monitoring apparatus 100 may include a respiratory waveform detection module 102. Respiratory waveform detection module 102 is used to perform non-contact respiratory monitoring of monitored subject 120 and to generate a waveform representing the monitored respiratory process. A number of different techniques to perform the non-contact monitoring may be used and are described in greater detail below. In one embodiment, biofeedback and monitoring apparatus 100 is a portable device.

Biofeedback and monitoring apparatus 100 may also include a cardiac waveform detection module 104. Cardiac waveform detection module 104 is used to perform non-contact cardiac monitoring of monitored subject 120 and to generate a waveform representing the monitored cardiac process. A number of different techniques to perform the non-contact cardiac monitoring may be used and are described in greater detail below.

Once a waveform representing the monitored respiratory or cardiac function has been generated, biofeedback and monitoring system 10 analyzes the generated waveform to determine whether the current monitored physiologic process is optimal. In one embodiment, the generated waveform is programmatically analyzed by a software analysis module 132 executing on a computing device 130. Computing device 130 may take many forms, including but not limited to a personal computer, workstation, server, network computer, quantum computer, optical computer, bio computer, Internet appliance, mobile phones and other mobile devices such as smartphones, a pager, a tablet computing device, or other form of computing device equipped with a processor and able to execute analysis module 132. Computing device 130 may be electronic and may include a Central Processing Unit (CPU), memory, storage, input control, modem, network interface, etc. The CPU may control each component of computing device 130 to provide an environment suitable for executing analysis module 132. The memory on computing device 130 temporarily stores instructions and data and provides them to the CPU so that the CPU operates the computing device 130.

Optionally, computing device 130 may include multiple CPUs for executing software loaded in memory and other programs for controlling system hardware. Each of the CPUs can be a single or a multiple core processor. The code loaded in the memory may run in a virtualized environment, such as in a Virtual Machine (VM). Multiple VMs may be resident on a single processor. Also, part of the code may be run in hardware, for example, by configuring a field programmable gate array (FPGA), using an application specific instruction set processor (ASIP) or creating an application specific integrated circuit (ASIC).

Input control for the computing device 130 may interface with a keyboard, mouse, microphone, camera, such as a web camera, or other input devices such as a 3D mouse, space mouse, multipoint touchpad, accelerometer-based device, gyroscope-based device, etc. Computing device 130 may receive, through the input control, input data relevant for calculating target waveforms for monitored subject 120. Optionally, computing device 130 may display data relevant to the generated waveform on a display as part of the analysis process.

In one embodiment, biofeedback and monitoring apparatus 100 communicates with computing device 130 over a network 110. Network 110 may be the Internet, an intranet, LAN (Local Area Network), WAN (Wide Area Network), MAN (Metropolitan Area Network), wireless network or some other type of network over which biofeedback and monitoring apparatus 100 and computing device 130 can communicate. Although depicted as a separate device in FIG. 1, it should also be appreciated that computing device 130 may be part of an integrated apparatus with biofeedback and monitoring apparatus 100.

Ultrasound probe 101 uses ultrasonic energy to detect cardiac and/or respiratory function in a monitored subject. Ultrasound probe 101 illuminates a monitored subject with ultrasonic waves and tracks how long the returned waves take to reflect back to the probe. In some embodiments, measurements may be taken over 100 times per second. The gathered data may be communicated to biofeedback and monitoring apparatus 100 in a number of different ways. For example, ultrasound probe may communicate via an earphone jack, microphone port, through a multi-pin port or dock connector, through a USB port, via BLUETOOTH (if the probe is equipped with its own power source such as a battery) or some other type of communication interface. In one embodiment, ultrasound probe 101 may be connected to a smart phone via a microphone port without disrupting the audio capabilities of the smartphone. Once captured, the data is analyzed by analysis module 132. Since sound travels at known speed, Time of Flight measurement is used to create waveforms and analyze chest movements of subjects to identify cardiac and respiratory function.

The non-contact monitoring system uses ultrasound probe 101 emitting ultrasonic waves to identify cardiac and respiratory waveforms in patients. The ultrasound probe 101 transmits ultrasonic waves towards a subject and then detects the sound waves reflected from the subject that are indicative of respiratory and/or cardiac functions. The detected reflections are used to plot a two-dimensional waveform. The waveforms represent the rise and fall of a detected signal (the reflected sound waves) over time and are indicative of the small movements of the patient's chest, abdomen and/or other anatomical sites that are associated with respiratory and/or cardiac function.

As noted above, the ultrasound probe emits ultrasonic energy to gather data for the monitoring system. Ultrasonic sound is a vibration at a frequency above the range of human hearing, in other words usually in a range above 20 kHz. A transducer in the ultrasound probe 101 radiates a beam of ultrasound to illuminate a subject patient. In one embodiment, the radiated beam of ultrasound is for example in the 25 kHz to 500 kHz range. A receiving transducer in the ultrasound probe 101 detects the signals reflected from the monitored subject which shift slightly from the incident frequency due to cardiac or respiratory motion. In one embodiment, ultrasound probe 101 may include one or two Massa probes with associated peak detection, digitization, and communications hardware and firmware. The signal is then analyzed by a cardiac or respiratory detection module (102 or 104) and plotted to generate a waveform, which may be compared by the analysis module 132 against an appropriate benchmark. Appropriate adjustments are made by the monitoring system to account for the distance between the monitoring system and the subject as well as any environmental factors affecting the detection of the reflected energy. Additional specifications for exemplary embodiments of ultrasound probe 101 are described further below.

In an embodiment, the probe 101 may be one or more OEM Massa M5000, single membrane, 220 kHz ultrasound probes. The probe 101 may be capable of internally triggered pulse generation and detection at 100 Hz (maximum internal probe setting). The probe 101 may or may not perform sample averaging and may include a built-in peak detection algorithm. The probe may include a digital (serial, RS485) data output to an interface board.

An exemplary interface board in communication with the probe 101 may assist in data acquisition and may peak detect output buffered on probes and sampled at 100 Hz (each probe) over RS485 probe interface and support 200 Hz DAQ, 16 bit range data and ˜3.2 kbps stream of 2 channel distance data. The exemplary interface board may also assist in data processing by appending real-time clock (RTC) data to the signal data stream at specified intervals and using interface board memory for buffering data as necessary. The interface board also assists in uploading the signal data to the computing device 130. For example, in one embodiment, the data is transferred to the computing device 130 via a USB connection with the computing device being responsible for USB bus inquiry and data storage.

In one embodiment, the interface board sits between the ultrasound probes and the computing device and manages communication with both the probes and computing device. FIG. 1A depicts an exemplary interface board. The interface board may contain a real time clock 151, power management circuitry 152, a microcontroller 153 programmed with system logic firmware, a USB-RS485 interface and memory 155 for storage of data and configuration information.

The power management subsystem 152 of an exemplary interface board is responsible for maintaining voltage levels required for powering the probe(s) and for powering other components on the interface board itself. In one configuration, all power is supplied by the USB port of a computing device. The interface board may also include an external DC input port for contingency power (due to variations/unknowns in laptop USB performance and the existence of multiple probes) and to enable stand-alone (without a PC) data acquisition. In one embodiment, power management consists of two regulated supplies—one for the interface electronics (3V) and one for the Massa probe (15V step-up from USB 5V nominal supply).

The exemplary interface board may also include a microcontroller 153, real-time clock 151, and memory block 155. The microcontroller 153 manages low-level aspects of device performance including probe configuration, memory management, probe-side data request/reading, PC-side data send, clock functions, etc. The real-time clock 151 is used to ensure known sampling intervals and to allow periodic time stamping of acquired data. Accurate sampling rate information is required for estimation of respiratory rate. Periodic time stamps are utilized in offline processing of data for correlation with other data streams and events. The memory block 155 on the exemplary interface board 150 may be utilized for storage of configuration information, caching of data prior to sending to the computing device 130, stand-alone data acquisition, etc.

One example of a suitable non-contact monitoring system that may be leveraged in conjunction with the embodiments of the present invention is described in U.S. Pat. No. 6,062,216 ('216 patent). As described in the '216 patent, a respiratory monitor may employ either ultrasonic or laser monitoring of an individual's breathing function by measuring changes in body position with respect to time. The device continuously and without the need for contact, monitors the individual's breathing function (and analyzes the measured waveform and identifies respiratory rate, apneic pauses, and obstructive breathing) and body movements. The '216 patent (the contents of which are hereby incorporated by reference) describes a monitoring system using ultrasonic energy to monitor respiratory function so as to detect sleep apnea but may be adapted to perform the respiratory monitoring described herein. It should be appreciated that although the monitoring system of the '216 patent has been cited as an exemplary monitoring system which may be used in the present invention, other non-invasive monitoring systems utilizing ultrasonic energy to detect respiratory waveforms may also be used within the scope of the present invention.

Once captured, the ultrasound signals must be processed in order to be analyzed. In one embodiment, the processing begins by pre-processing the raw signal. The pre-processing assesses the performance of the raw signal measurement and determines whether the incoming signal should be processed further or flagged as poor quality. This step is performed on the raw waveform after Analog/Digital (A/D) conversion. Problems with transducer function, lack of a detectable echo, or a target that is too close to the transducer are primary targets of the diagnostic routines contained in this step.

Once it is determined that the raw data can be processed further, in one embodiment a distance/velocity estimation subsystem processes the data to determine a combination of the distance to the target and the change in that distance from the last measurement (velocity). These estimates may be made using basic thresholding techniques, advanced approaches such as correlation analysis, etc. In one embodiment, the optimal solution consists of a combination of multiple processing approaches.

In one embodiment, the estimated signal is processed by a filtering subsystem. The estimation of distance from the raw data results in a signal that contains various types of noise. Since the movement of the body due to respiration is a bandwidth-limited and range-limited process, the estimated data is filtered to remove components that can not be due to the underlying physiology. This subsystem implements a digital filter that smoothes the distance estimate enabling better parameterization and display of the waveform.

Upon the completion of filtering, in one embodiment the signal is handled by a post-processing subsystem. After filtering of the distance measurement, the waveform should resemble a smooth pseudo-sinusoidal signal representative of breathing. If it does not, then this is a strong indication that respiration is not being measured or is being measured poorly. The post-processing QOS subsystem attempts to flag waveforms that are likely not accurate measures of respiration so that important parameters will not be calculated from those data and important decisions will not be based on the data.

Analysis module 132 analyzes the generated waveform produced by biofeedback and monitoring apparatus 100. The generated waveform may be programmatically compared against stored waveform patterns 134 to determine whether the current generated waveform represents an optimal waveform for the monitored physiologic process. The selection of the comparison waveform from the stored waveform patterns may utilize previous input data 136 that includes information regarding the monitored subject such as personal medical information (e.g. sex, height, weight, age, family history of various diseases, etc. and occupational information). Based on available data, the analysis module 132 selects either a customized target waveform or a default waveform for comparison to the generated waveform.

In one embodiment, the analysis of the generated waveform may be a programmatic process that occurs in an automated fashion. In an alternate embodiment, the process may also involve human input in reviewing the selection of the target waveform prior to completion of the analysis. In one embodiment, all of the analysis decisions are saved for future study in order to continually refine the stored waveform patterns 134.

For some monitored conditions it is useful to provide biofeedback to a monitored subject to help the subject achieve or maintain a desired waveform. In such a case, the results of the analysis performed by the analysis module 132 are provided to biofeedback module 106. It should be appreciated that in some embodiments, the functionality attributed to the various modules in the present invention may be combined into a single module or split into additional modules without departing from the scope of the present invention. Depending upon the results of the analysis, biofeedback module 106 may take a number of actions. If the monitored subject is already exhibiting a waveform for the monitored physiologic process consistent with a desired target waveform, biofeedback module 106 may provide limited or no biofeedback. Instead, the biofeedback module 106 may provide alternative sensory feedback designed to create an environment conducive to maintaining the current respiratory or cardiac function. For example, biofeedback module 106 may provide only audible feedback such as music via audio module 140 or aromatic feedback via aroma dispensing module 144 designed to maintain the status quo. Alternatively, in such a situation, biofeedback module 106 may provide no feedback at all as the monitored subject has already achieved a desired waveform.

If the results of the analysis performed by analysis module 132 show a discrepancy between the generated waveform (representing the monitored physiologic process) and the target waveform selected by the analysis process, the biofeedback module may provide biofeedback such as providing biofeedback via a display 142 visible to monitored subject 120. The biofeedback may include graphical representations of the generated waveform and target waveform and numerical values representing current physiological measurements. In one embodiment, the two waveforms may be superimposed over each other. Biofeedback module 106 may also provide numerical data such as current respiratory and/or cardiac rates and visual instructions to monitored subject 120 suggesting monitored subject take a particular action (e.g.: begin a slow breathing exercise to attempt to control respiration rate) that will move the subject towards the target waveform.

In one embodiment, the analysis module 132 may report a significant discrepancy between the generated waveform of the monitored physiological process and the target waveform that exceeds a pre-determined parameter. In such a circumstance, biofeedback module 106 may provide an intermediate waveform to monitored subject 120 rather than the target waveform in an attempt to incrementally adjust the monitored physiological process. The intermediate waveform in such a situation may represent a more attainable goal to monitored subject 120 and its use may prevent the monitored subject from becoming alarmed (which is counter-productive) over the size of the difference between the generated and target waveforms. Biofeedback module 106 may provide a number of intermediate waveforms as appropriate for the monitored subject to attempt to replicate as part of the biofeedback in order to incrementally move the monitored subject towards his or her target waveform. The embodiments of the present invention thus provide the ability to adjust real-time non-contact biofeedback based on the subject's actual response to the intervention. This method is consistent with the movement to personalized medicine where interventions are made specific to a user, not just a population.

In one embodiment the biofeedback module 106 displays the breathing waveform derived from the non-contact measurement of breathing. Of note, the breathing waveform can be captured through clothes and does not need a specific window to receive the necessary information to generate a breathing or cardiac waveform. However, in one embodiment, a signal enhancer 122 may be utilized to augment the reflected signal. This may be in the form of a “relaxation patch” worn by the participant.

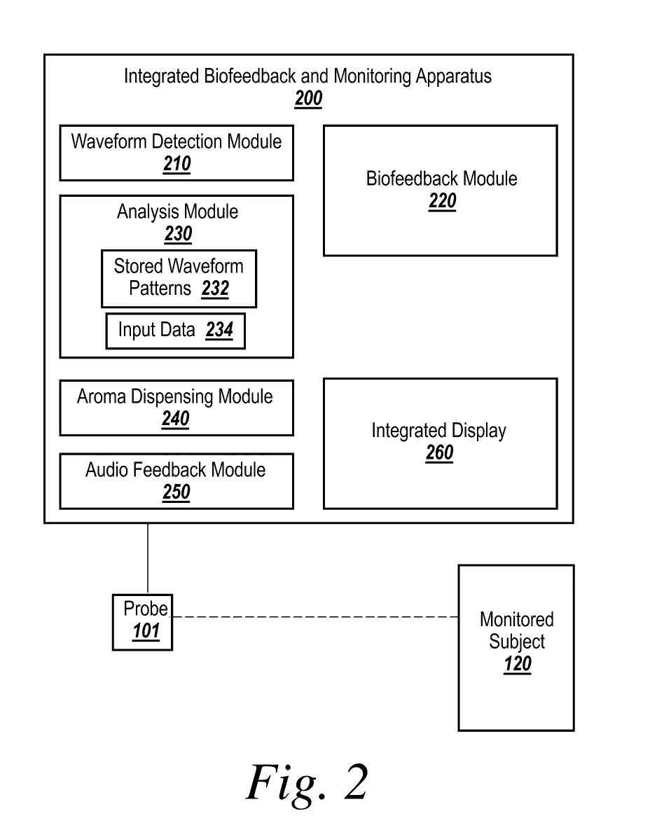

In one embodiment, the biofeedback and monitoring system described herein may be provided as an integrated biofeedback and monitoring apparatus rather than as separate components in multiple devices. FIG. 2 depicts an exemplary integrated biofeedback apparatus 200 that includes most or all of the components of the biofeedback and monitoring system described in FIG. 1. The integrated biofeedback apparatus 200 may include one or more waveform detection modules 210 such as respiratory waveform detection modules and cardiac waveform detection modules. Ultrasound probe 201 detects respiratory and cardiac function in a monitored subject and communicates the gathered data to integrated biofeedback apparatus 200. The integrated biofeedback apparatus 200 may also include biofeedback module 220 and analysis module 230. It will be appreciated that biofeedback module 220 and analysis module 230 may be combined into a single module or split into additional modules without departing from the scope of the present invention.

Analysis module 230 may include stored waveform patterns 232 and stored input data 234 specific to a monitored subject. In one embodiment, integrated biofeedback apparatus 200 may also include an aroma dispensing module 240 an audio module 250 for providing aromatic and audio feedback and an integrated display module 260 utilized to provide biofeedback to a monitored subject in the manner described herein. In some embodiments, aroma dispensing module 240 and audio module 250 may communicate with additional devices in proximity to the monitored subject in order to perform their function. For example, the modules may communicate in a wired or wireless manner with additional devices to provide audio or aromas for the monitored subject. In other embodiments, integrated biofeedback apparatus 200 may contain some but not all of the modules 240, 250 and 260 used to provide feedback and biofeedback. The aroma dispensing module 240 may include one or more stored scents that are designed to be soothing when inhaled and that are released into the monitored subject's environment upon a signal received from the biofeedback module 206.

In one exemplary embodiment, the Integrated Biofeedback and Monitoring Apparatus 200 may be provided via a portable device such as a mobile phone, laptop or tablet computing device. In such a case, the ultrasound probe may communicate via an earphone jack, microphone port, through a multi-pin port or dock connector, through a USB port, via BLUETOOTH (if the probe is equipped with its own power source such as a battery) or some other type of communication interface. The gathered data is analyzed by the analysis module.

In an embodiment, Integrated Biofeedback and Monitoring Apparatus 200 may include some or all of the following: a power supply and regulation subsystem, an ultrasound transducer, a high voltage generation subsystem, an analog signal filtering subsystem, an analog to digital conversion component, a digital signal processor (DSP) element and a system control subsystem.

An exemplary power supply and regulation subsystem connects to and regulates an electrical power source. The input is a DC voltage. The output is one or more regulated DC voltages to be used for powering other module subsystems. For some applications and depending on electrical component selection this subsystem may be reduced to an AC adapter that plugs into the wall socket and into the module.

An exemplary ultrasound transducer (or transmit/receive pair) produces and detects ultrasonic energy. Transducers are selected for optimal frequency, beam angle, and power requirements. Transducers are may be activated by sinusoidal waveforms with an amplitude of tens to hundreds of volts. Echo signals that return to the transducer for detection may be three orders of magnitude weaker.

An exemplary high voltage generation subsystem may generate high voltage from available supply voltages in order to drive the transceiver output. This subsystem boosts voltage using a series of carefully selected passive electrical components: resistors, capacitors, and inductors. The input to this subsystem may be either a regulated DC supply voltage or a pulse-width modulated version of the supply voltage. The output is a high voltage with range and tolerance based on transducer selection.

An exemplary analog filtering subsystem includes a series of analog filter and amplification blocks build around operational or instrumentation amplifiers. The signal filters are tuned to preferentially pass the activation frequency of the transducer so that the echo signal is amplified relative to any other signals of noise being detected. The input of this subsystem is the very noisy low-voltage signal output of the transducer. The output is a filtered and conditioned version of the transducer output, ready for digitization. The filter subsystem may benefit from a dynamic component that monitors signal strength and adjusts subsystem gain to maximize use of A/D dynamic range.

An exemplary analog to digital conversion subsystem converts the filtered and signal conditioned transducer output to a quantized, time-sampled digital signal. While listed independently, this subsystem may be a built-in component of either the DSP or the control hardware components described below. Primary considerations when selecting an A/D converter are the maximum sampling rate and the digital dynamic range.

An exemplary DSP component may include a digital integrated circuit that is designed with an architecture that supports rapid processing of sampled data. The DSP component can perform much of the intensive signal processing more rapidly than a standard microcontroller. The amount of incoming data and the complexity of the signal processing in some embodiments may utilize a dedicated DSP component. In other embodiments however, a dedicated DSP may not be required and the processing burden may be handled by a microcontroller. The DSP component may have a built-in A/D converter.

An exemplary system control subsystem includes a microcontroller responsible for system-level logic, monitoring ongoing processes, performing calculations, signal processing, and communications. The microcontroller and the DSP subsystem may work in concert in certain embodiments. In some embodiments the DSP or microcontroller or both may be eliminated for cost and complexity savings. The microcontroller may contain a built-in A/D converter.

The biofeedback module and analysis modules described herein may be pre-installed or downloaded to the device. The phone or laptop display and speakers may be used to provide visual biofeedback and audio feedback respectively.

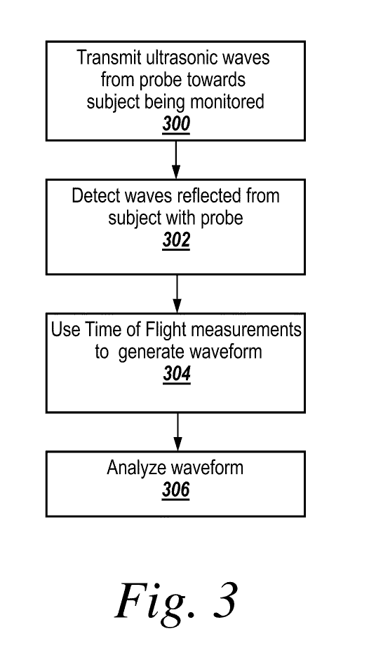

FIG. 3 depicts an exemplary sequence of steps performed by an embodiment of the present invention to gather data using the ultrasound probe. The sequence may begin with the transmission of ultrasonic waves by the ultrasound probe towards a monitored subject in order to detect cardiac and/or respiratory motion (step 300). The probe then detects waves reflected from the monitored subject (step 302). Time of Flight calculations are performed on the gathered data and a waveform is generated (step 304). The generated waveform may then be analyzed (step 306).

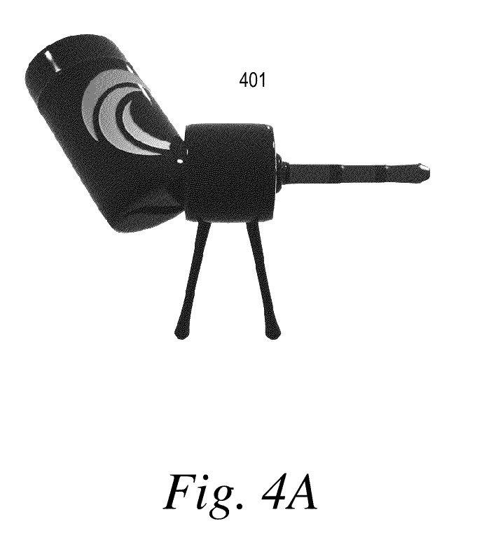

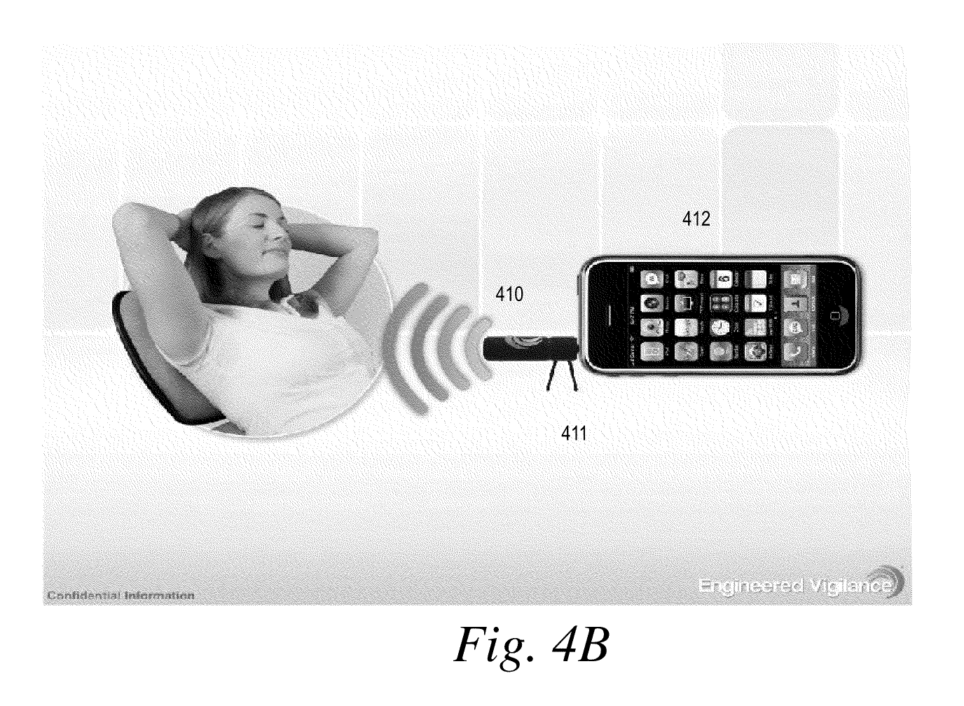

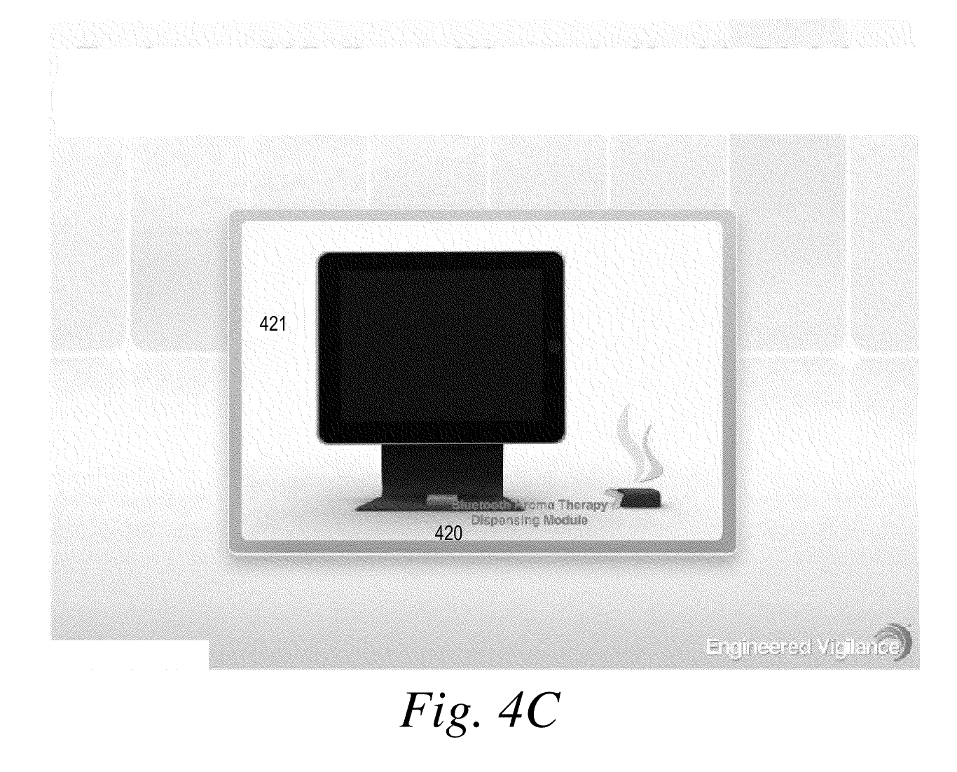

FIGS. 4A-4C depict the ultrasound probe. FIG. 4A depicts an exemplary ultrasound probe 401. The probe may draw power from an attached device through a physical connection or may have a power source independent from the monitoring device with which the probe is communicating. In one embodiment, the probe is attached to a supporting base in a manner that allows the probe to be adjusted or swiveled by a user to project in another direction without moving the base. FIG. 4B depicts an exemplary ultrasound probe 410 with a hinged stand 411 connected to a smart phone 412 (not drawn to scale). In one embodiment, a hinged stand for the probe enables landscape viewing to be activated for an attached IPHONE. In another embodiment, the stand is adjustable. In other embodiments the stand may be adjustable but not hinged or not adjustable or hinged. FIG. 4C depicts an exemplary ultrasound probe 420 built into a stand for a tablet computing device 421. For example, in one embodiment, the built-in probe is connected to a stand for an IPAD. In one embodiment, the probe may be built into the base of the stand. In another embodiment the probe may be included in the stand in a location other than the base.

It should be understood that other physiologic parameters, (video, audio, etc) could be incorporated to add robustness to the proposed system. Further, though breathing, cardiac and body movement are optimally derived through non-contact means to prevent the creation of an overly artificial environment, a contact monitoring system may also be used to perform monitoring of a subject.

Some or all of the present invention may be provided as one or more computer-readable programs embodied on or in one or more non-transitory physical mediums. The mediums may be a floppy disk, a hard disk, a compact disc, a digital versatile disc, a flash memory card, a PROM, an MRAM, a RAM, a ROM, or a magnetic tape. In general, the computer-readable programs may be implemented in any programming language. Some examples of languages that can be used include C, C++, C#, Python, FLASH, JavaScript, or Java. The software programs may be stored on, or in, one or more mediums as object code. Hardware acceleration may be used and all or a portion of the code may run on a FPGA, an Application Specific Integrated Processor (ASIP), or an Application Specific Integrated Circuit (ASIC). The code may run in a virtualized environment such as in a virtual machine. Multiple virtual machines running the code may be resident on a single processor.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

In addition, implementations consistent with principles of the invention can be implemented using devices and configurations other than those illustrated in the figures and described in the specification without departing from the spirit of the invention. Devices and/or components may be added and/or removed from the implementations described herein depending on specific deployments and/or applications.

We claim:

1. An ultrasound probe apparatus for use with a portable monitoring system performing non-contact monitoring of respiratory function, comprising:
transmission means for transmitting ultrasonic waves towards a monitored subject, the waves transmitted in a range above 20 kHz;
receiving means for receiving reflected waves bouncing off of the monitored subject;
an interface for transmitting data regarding the transmitted and received ultrasonic waves to a monitoring system executing on a portable device equipped with a processor, the monitoring system including a waveform detection module for generating a waveform from the data, the portable device including, or in communication with, an analysis module for programmatically analyzing the generated waveform to identify a respiratory function of the subject.

2. The ultrasound probe apparatus of claim 1 wherein the portable device is a smartphone.

3. The ultrasound probe apparatus of claim 1 wherein the portable device is a tablet computing device or laptop computing device.

4. The ultrasound probe apparatus of claim 1 wherein the ultrasound probe apparatus is physically connected to a smartphone, a tablet computing device or a laptop.

5. The ultrasound probe apparatus of claim 1, further comprising:
a hinged stand, the hinged stand providing physical support of the ultrasound probe apparatus.

6. The ultrasound probe apparatus of claim 5 wherein the hinged stand provides support for the ultrasound probe apparatus and an attached smartphone.

7. The ultrasound probe apparatus of claim 1 wherein the ultrasound probe apparatus is built into a stand providing physical support for a tablet computing device.

8. The ultrasound probe apparatus of claim 1 wherein the interface is at least one of a USB interface, BLUETOOTH interface, multipin port interface or audio port interface.

9. The ultrasound probe apparatus of claim 1 wherein the direction of the transmission means is manually adjustable by a user.

10. The ultrasound probe apparatus of claim 1 further comprising:
an independent power source for powering the ultrasound probe apparatus.

11. A portable monitoring system performing non-contact monitoring of respiratory function, comprising:
an ultrasound probe transmitting ultrasonic waves towards a monitored subject, and receiving reflected waves bouncing off of the monitored subject, the waves transmitted in a range above 20 kHz;
an interface for transmitting data regarding the transmitted and received ultrasonic waves to a portable device from the ultrasound probe; and
the portable device, the portable device equipped with a processor and including a waveform detection module, the waveform detection module receiving data about the transmitted and reflected waves from the probe and generating a waveform from the data, the portable device including, or in communication with, an analysis module for programmatically analyzing the generated waveform to identify a respiratory function of the subject.

12. The portable monitoring system of claim 11 wherein the portable device is a smartphone.

13. The portable monitoring system of claim 11 wherein the portable device is a tablet computing device or laptop computing device.

14. The portable monitoring system of claim 11 wherein the ultrasound probe apparatus is physically connected to a smartphone, a tablet computing device or a laptop.

15. The portable monitoring system of claim 11, further comprising:
a hinged stand providing physical support of the ultrasound probe.

16. The portable monitoring system of claim 11 wherein the ultrasound probe apparatus is built into a stand providing physical support for a tablet computing device.

17. The portable monitoring system of claim 11 wherein the interface is at least one of a USB interface, BLUETOOTH interface, multipin port interface or audio port interface.

18. The portable monitoring system of claim 11 further comprising:
an independent power source for powering the ultrasound probe.

19. A method for monitoring respiratory function using an ultrasound probe, comprising:
transmitting with an ultrasound probe ultrasonic waves towards a monitored subject in a range above 20 kHz;
receiving with the ultrasound probe reflected waves bouncing off of the monitored subject;
transmitting from the probe to a portable device the data regarding the transmitted and received ultrasonic waves, the data transmitted via a probe interface;
generating programmatically on the portable device a waveform from the data; and
analyzing programmatically the generated waveform to identify a respiratory function of the subject.

20. A physical computer-readable medium holding computer-executable instructions for monitoring respiratory function using an ultrasound probe, the instructions when executed causing one or more devices to:
transmit with an ultrasound probe ultrasonic waves towards a monitored subject in a range above 20 kHz;
receive with the ultrasound probe reflected waves bouncing off of the monitored subject;
transmit from the probe to a portable device the data regarding the transmitted and received ultrasonic waves, the data transmitted via a probe interface;
generate programmatically on the portable device a waveform from the data; and
analyze programmatically the generated waveform to identify a respiratory function of the subject.
